(19) United States
(12) Patent Application Publication
(10) Pub. No.: US 2019/0142470 A1
(43) Pub. Date: May 16, 2019

(54) SPINAL SCREW HOLDER FOR MINIMAL INVASIVE SURGERY

(71) Applicant: Mantiz Logitech Co., Ltd., Daegu (KR)

(72) Inventors: Hyeun Sung KIM, Seoul (KR); Dong hwa HEO, Seoul (KR); Hong won YOON, Busan (KR)

(21) Appl. No.: 16/189,066

(22) Filed: Nov. 13, 2018

(30) Foreign Application Priority Data
Nov. 14, 2017 (KR) 10-2017-0151500
Aug. 31, 2018 (KR) 10-2018-0103258

(51) Int. Cl. A61B 17/70

(52) U.S. Cl. CPC A61B 17/7007; A61B 17/00234; A61B 17/7037; A61B 17/7085

(57) ABSTRACT

Provided is a spinal screw holder for a minimal invasive surgery. The spinal screw holder includes a first joint part having both penetrated ends, which is detachably coupled to a screw head fixed to a vertebra so as to be exposed to a predetermined height through an opening formed in a skin of a subject and a second joint part having both penetrated ends, which is coupled to an upper end of the first joint part and which is linearly movable in a direction that is away from the upper end of the first joint part and rotates to be bent with respect to the upper end of the first joint part after the second joint part is inserted through the opening in a state of being disposed in a straight line together with the first joint part.

### CROSS-REFERENCE TO RELATED APPLICATIONS

This U.S. non-provisional patent application claims priority under 35 U.S.C. § 119 of Korean Patent Application Nos. 10-2017-0151500 filed on Nov. 14, 2017, and 10-2018-0103258 filed on Aug. 31, 2018, the entire contents of which are hereby incorporated by reference.

### BACKGROUND

The present invention relates to a spinal screw holder for a minimal invasive surgery, and more particularly, to a spinal screw holder for a minimal invasive surgery, in which an interference of a fixing rod is maximally avoided during a surgical procedure so as to be smoothly inserted into to be fixed to a screw head.

A disk functions as a joint between vertebrae and minimizes an impact applied to the vertebrae by changing a position and shape of nucleus pulposus according to movement of a spine.

Most of the nucleus pulposus are composed of moisture (water). As an amount of water contained in the nucleus pulposus gradually decreases with aging, the disk loses its buffering function to cause various spinal diseases accompanied with pain.

Thus, if the surgery for removing a severely deformed or damaged disc is performed, it is necessary to preferentially take measures for maintaining a gap between vertebrae and preventing the vertebrae from being deformed or shaken.

Here, spine-disk patients may be operated according to following procedures.

First, a disk corresponding to a damaged portion of the vertebra is removed so that the damaged portion of the vertebra is not pushed or pressed, and then, bone fragments are filled into a hollow artificial aid (cage) formed of a metal or plastic material, and the artificial aid is inserted into the portion of the vertebra from which the disk is removed.

Sequentially, a pedicle screw is inserted into each of portions of the vertebra, which correspond to upper and lower sides of the damaged disk, and then, a rod is connected to the pedicle screw to secure a distance between the vertebrae, thereby normally realizing osseointegration (hereinafter, referred to an operation 1).

If there is slight disc damage between the vertebrae of the spine-disk patients, the following procedure may be performed.

That is, the disk leaves as it is, and a pedicle screw is inserted into and fixed to each of portions of the vertebra, which correspond to upper and lower sides of the damaged disk, and then, a rod is connected to the pedicle screw to secure a distance between the vertebrae, thereby preventing the damaged disk from being worsen (hereinafter, referred to as an operation 2).

However, since the operations 1 and 2 essentially require a process of cutting a skin corresponding to the damaged vertebra by a predetermined size so as to couple the rod to the pedicle screw after the pedicle screw is coupled to the vertebra, an invasive portion may be large to cause limitations in which recovery of the patient is delayed, and satisfaction after the operation is low due to the wound.

As invented with this point in view, “a minimally invasive surgical screw holder having a joint and a minimally invasive surgical apparatus using the same” (hereinafter, referred to as a “prior art”) is disclosed in Korean Patent registration No. 10-1703003, which is applied and registered by the present applicant.

As illustrated in FIGS. 18A and 18B, the prior art includes a holder body 110 having both penetrated ends and a detachable part 120 rotatably coupled to an end of the holder body 110 and coupled to a head part 401 of a screw 400 fixed to the vertebra.

However, in the prior art, if the holder body 110 exposed on the skin through an opening of a subject is provided in plurality, when it is intended to align positions of rod insertion guide grooves 124 of the plurality of detachable parts 120 according to the body shape of the subject or the surgical environments, it is difficult to grasp the positions of the rod insertion guide grooves 124 of the detachable parts 120 inserted under the skin of the subject.

Thus, it is necessary to develop a device and a tool for allowing a rod inserted through the skin of the subject to be smoothly inserted into each of the rod insertion grooves 410 formed in a head 401 of each of the plurality of screws 400.

### SUMMARY

The prevent invention provides a spinal screw holder for a minimal invasive surgery, in which an interference of a fixing rod is maximally avoided during a surgical procedure so as to be smoothly inserted into and fixed to a screw head.

Also, the present invention provides a spinal screw holder for a minimal invasive surgery, in which a second joint part is not shaken when a lower end of the second joint part, which is disposed at a relatively upper side, of two joint parts constituting the spinal screw holder is fitted to be coupled to an upper end of a first joint part disposed at a relatively lower side so that the surgery is quickly and stably performed when lordosis of a vertebra is formed by closely attaching the spinal screw holders to each other after the two joint parts are coupled to each other by an operator.

An embodiment of the present invention provides a spinal screw holder for a minimal invasive surgery, including: a first joint part having both penetrated ends, which is detachably coupled to a screw head fixed to a vertebra so as to be exposed to a predetermined height through an opening formed in a skin of a subject; and a second joint part having both penetrated ends, which is coupled to an upper end of the first joint part and which is linearly movable in a direction that is away from the upper end of the first joint part and rotates to be bent with respect to the upper end of the first joint part after the second joint part is inserted through the opening in a state of being disposed in a straight line together with the first joint part, wherein the upper end of the first joint part has an outer diameter or width that is equal to or greater than an inner diameter or width of the lower end of the second joint part, the first joint part further includes: a first cutoff slit having a predetermined width and cut by a predetermined length downward from an edge of the upper end of the first joint part; and a second cutoff slit which has a predetermined width and is cut by a predetermined length downward from the edge of the upper end of the first joint part and which is disposed in parallel to face the first cutoff slit, and each of both left and right sides of the upper end of the first joint part have a diameter or width that is variable within a predetermined range with respect to the first cutoff slit and the second cutoff slit.

Another embodiment of the present invention provides a spinal screw holder for a minimal invasive surgery, including: a first joint part having both penetrated ends, which is detachably coupled to a screw head fixed to a vertebra so as to be exposed to a predetermined height through an opening formed in a skin of a subject; and a second joint part having both penetrated ends, which is coupled to an upper end of the first joint part and which is linearly movable in a direction that is away from the upper end of the first joint part and rotates to be bent with respect to the upper end of the first joint part after the second joint part is inserted through the opening in a state of being disposed in a straight line together with the first joint part, wherein the bending of the second joint part at an angle of about 90 degrees or less in both left and right directions with respect to the upper end of the first joint part is restricted after moving in the direction that is away from the upper end of the first joint part in the state of being disposed in the straight line together with the first joint part, and the second joint part further includes: a rotation-allowable cutoff surface cut in a flat shape from the edge of the upper end of the first joint part and formed on each of both outer surfaces of the upper end of the first joint part; and an excessive rotation restriction protrusion protruding to be stepped on each of both sides of an edge of a lower end of the rotation-allowable cutoff surface, wherein, when the second joint part rotates with respect to the upper end of the first joint part, an edge of one side of each of the rotation pieces extending from both sides of the edge of the lower end of the second joint part contacts the excessive rotation restriction protrusion so that the second joint part is perpendicular to the first joint part.

Further another embodiment of the present invention provides a spinal screw holder for a minimal invasive surgery, including: a first joint part having both penetrated ends, which is detachably coupled to a screw head fixed to a vertebra so as to be exposed to a predetermined height through an opening formed in a skin of a subject; a second joint part having both penetrated ends, which is coupled to an upper end of the first joint part and which is linearly movable in a direction that is away from the upper end of the first joint part and rotates to be bent with respect to the upper end of the first joint part after the second joint part is inserted through the opening in a state of being disposed in a straight line together with the first joint part; a rotation-allowable guide groove vertically recessed by a predetermined length in each of both sides of an upper portion of an outer surface of the first joint part; rotation pieces respectively extending from both sides of an edge of a lower end of the second joint part to face each other; a rotation pin inserted and fixed from a through-hole passing through each of the rotation pieces and accommodated in the rotation-allowable guide groove; and a linear movement guide protrusion disposed on each of both sides of the rotation piece in a longitudinal direction of a main body of the first joint part so that the rotation piece linearly moves without rotating when the second joint part moves downward to be coupled.

### BRIEF DESCRIPTION OF THE FIGURES

The accompanying drawings are included to provide a further understanding of the inventive concept, and are incorporated in and constitute a part of this specification. The drawings illustrate exemplary embodiments of the inventive concept and, together with the description, serve to explain principles of the inventive concept. In the drawings:

FIG. 1 is a perspective view illustrating a state in which a spinal screw holder for a minimal invasive surgery is coupled to a screw head according to an embodiment of the present invention;

FIGS. 2A-2C are views illustrating an overall structure of a first joint part that is a main part of the spinal screw holder for the minimal invasive surgery according to an embodiment of the present invention, wherein FIG. 2A is a perspective view, FIG. 2B is a conceptual side view when viewed from a viewpoint IIb of FIG. 2A, and FIG. 2C is a conceptual side view when viewed from a viewpoint IIc of FIG. 2A;

FIGS. 3A-3C are views illustrating an overall structure of a second joint part that is a main part of the spinal screw holder for the minimal invasive surgery according to an embodiment of the present invention, wherein FIG. 3A is a perspective view, FIG. 3B is a conceptual side view when viewed from a viewpoint IIIb of FIG. 3A, and FIG. 3C is a conceptual side view when viewed from a viewpoint IIIc of FIG. 3A;

FIG. 4 is an exploded perspective view illustrating an overall coupling relationship of the spinal screw holder for the minimal invasive surgery according to an embodiment of the present invention;

FIGS. 5A-5C are views sequentially illustrating a procedure using the spinal screw holder for the minimal invasive surgery according to an embodiment of the present invention, wherein FIG. 5A is a conceptual view illustrating a state in which the first joint part and the second joint part, which are disposed in a straight line, are inserted, FIG. 5B is a conceptual view illustrating a state in which the second joint part is pulled in a direction that is away from an upper end of the first joint part, and FIG. 5C is a conceptual view illustrating a state in which the second joint part rotates to be bent with respect to the upper end of the first joint part;

FIGS. 6 to 8 are perspective views sequentially illustrating a procedure in which a rod is coupled in a state in which a spinal screw holder for a minimal invasive surgery is inserted through a skin of a subject according to an embodiment or another embodiment of the present invention;

FIG. 9 is a perspective view illustrating a state in which the spinal screw holder for the minimal invasive surgery is coupled to a screw head according to another embodiment of the present invention;

FIGS. 10A and 10B are views illustrating a state in which the spinal screw holder for the minimal invasive surgery is coupled to the screw head according to another embodiment of the present invention, wherein FIG. 10A is a front view, and FIG. 10B is a cross-sectional view;

FIG. 11 is a side view illustrating a state in which the spinal screw holder for the minimal invasive surgery is coupled to the screw head according to another embodiment of the present invention;

FIG. 12 is a conceptual view illustrating a state in which a second joint part constituting the spinal screw holder for the minimal invasive surgery moves in a direction that is away from the first joint part to separate a lower end of the second joint part from an upper end of the first joint part in FIG. 11;

FIGS. 13A-13C are partial enlarged views illustrating a main part of the spinal screw holder for the minimal invasive surgery according to another embodiment of the present invention, wherein FIG. 13A is a view illustrating an upper end of the first joint part, FIG. 13B is a view illustrating a state in which the second joint part is coupled to the first joint part, and FIG. 13C is a partial enlarged view illustrating a state in which the second joint part and the second joint part are coupled to each other;

FIG. 14 is a partial enlarged view illustrating a main part of the spinal screw holder for the minimal invasive surgery according to another embodiment of the present invention, wherein the first view illustrates a state in which the second joint part is coupled to the first joint part when viewed at an oblique angle, and the second view illustrates a state in which the second joint part is coupled to the first joint part when viewed at an oblique angle;

FIG. 15A and FIG. 15B are views illustrating a state in which the first joint part and the second joint part, which constitute the spinal screw holder for the minimal invasive surgery, are separated from each other, wherein FIG. 15A is a front view, and FIG. 15B is a cross-sectional view;

FIG. 16A and FIG. 16B are views illustrating a state in which the first joint part and the second joint part, which constitute the spinal screw holder for the minimal invasive surgery, are separated from each other, wherein FIG. 16A is a side view, and FIG. 16B is a cross-sectional view;

FIGS. 17A-17D are views sequentially illustrating a procedure using the spinal screw holder for the minimal invasive surgery according to another embodiment of the present invention, wherein FIG. 17A is a conceptual view illustrating a state in which the first joint part and the second joint part, which are disposed in a straight line, are inserted, FIG. 17B is a conceptual view illustrating a state in which the second joint part is pulled in a direction that is away from an upper end of the first joint part, and FIG. 17C is a conceptual view illustrating a state in which the second joint part rotates to be bent at a predetermined angle with respect to the upper end of the first joint part, and FIG. 17D is a conceptual view illustrating a state in which the second joint part completely rotates to be bent with respect to the upper end of the first joint part; and

FIGS. 18A and 18B are conceptual views illustrating a structure of a spinal screw holder according to a related art.

### DETAILED DESCRIPTION

Advantages and features of the present disclosure, and implementation methods thereof will be clarified through following embodiments described with reference to the accompanying drawings.

The present invention may, however, be embodied in different forms and should not be construed as limited to the embodiments set forth herein.

In this specification, these embodiments are provided so that this disclosure will be thorough and complete, and will fully convey the scope of the present invention to those skilled in the art.

Also, the present invention is only defined by scopes of claims.

Accordingly, in some embodiments, well-known components, well-known device operations, and well-known techniques will not be described in detail to avoid ambiguous interpretation of the present invention.

Also, like reference numerals refer to like elements throughout. In the following description, the technical terms are used (mentioned) only for explaining a specific exemplary embodiment while not limiting the present disclosure.

The terms of a singular form may include plural forms unless referred to the contrary. The meaning of “include,” “comprise,” “including,” or “comprising,” specifies a component and an operation but does not exclude other components and operations.

Unless terms used in the present invention are defined differently, all terms (including technical and scientific terms) used in this specification have the same meaning as generally understood by those skilled in the art.

Also, unless defined apparently in the description, the terms as defined in a commonly used dictionary are not ideally or excessively construed as having formal meaning.

Hereinafter, preferred embodiments of the present invention will be described in more detail with reference to the accompanying drawings.

### First Embodiment

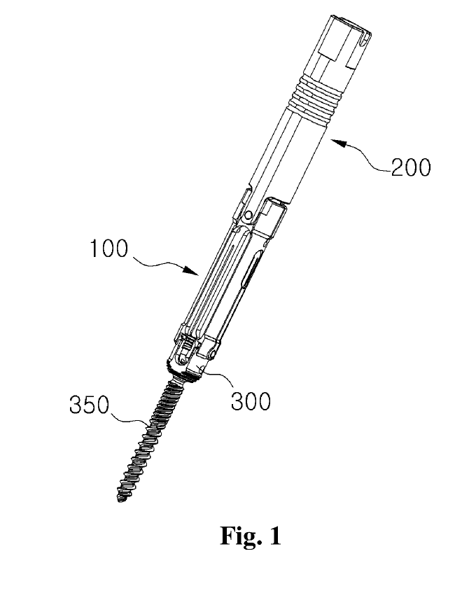

First, FIG. 1 is a perspective view illustrating a state in which a spinal screw holder for a minimal invasive surgery is coupled to a screw head according to an embodiment of the present invention.

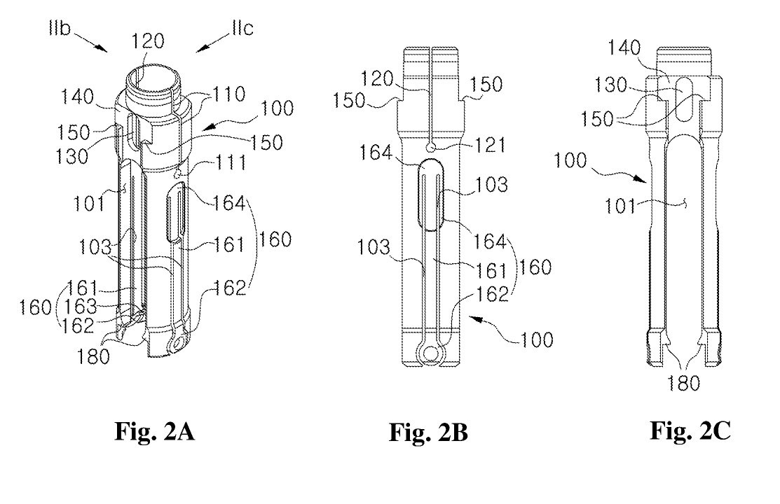

Also, FIGS. 2A-2C are views illustrating an overall structure of a first joint part that is a main part of the spinal screw holder for the minimal invasive surgery according to an embodiment of the present invention, wherein FIG. 2A is a perspective view, FIG. 2B is a conceptual side view when viewed from a viewpoint IIb of FIG. 2A, and FIG. 2C is a conceptual side view when viewed from a viewpoint IIc of FIG. 2A.

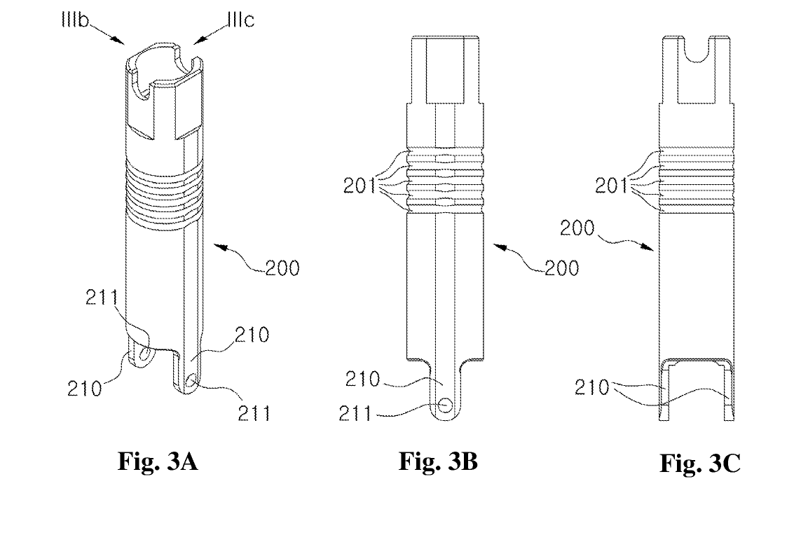

Also, FIGS. 3A-3C are views illustrating an overall structure of a second joint part that is a main part of the spinal screw holder for the minimal invasive surgery according to an embodiment of the present invention, wherein FIG. 3A is a perspective view, FIG. 3B is a conceptual side view when viewed from a viewpoint IIb of FIG. 3A, and FIG. 3C is a conceptual side view when viewed from a viewpoint IIc of FIG. 3A.

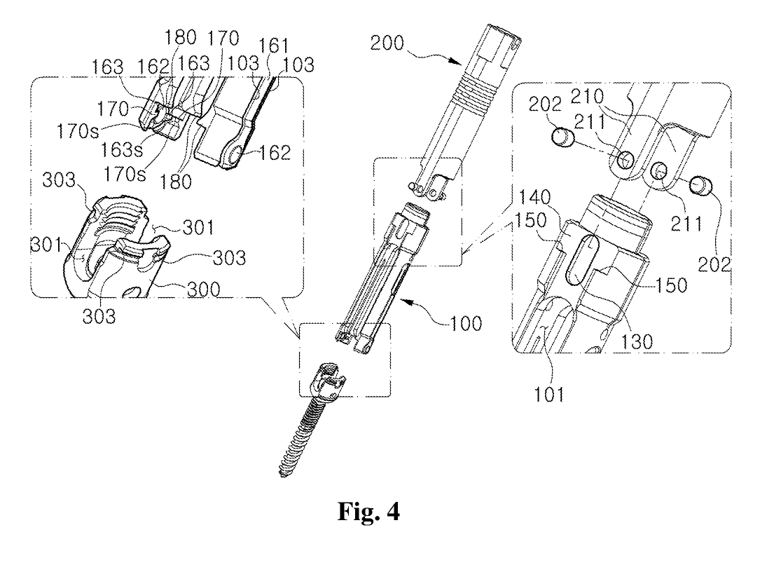

Also, FIG. 4 is an exploded perspective view illustrating an overall coupling relationship of the spinal screw holder for the minimal invasive surgery according to an embodiment of the present invention.

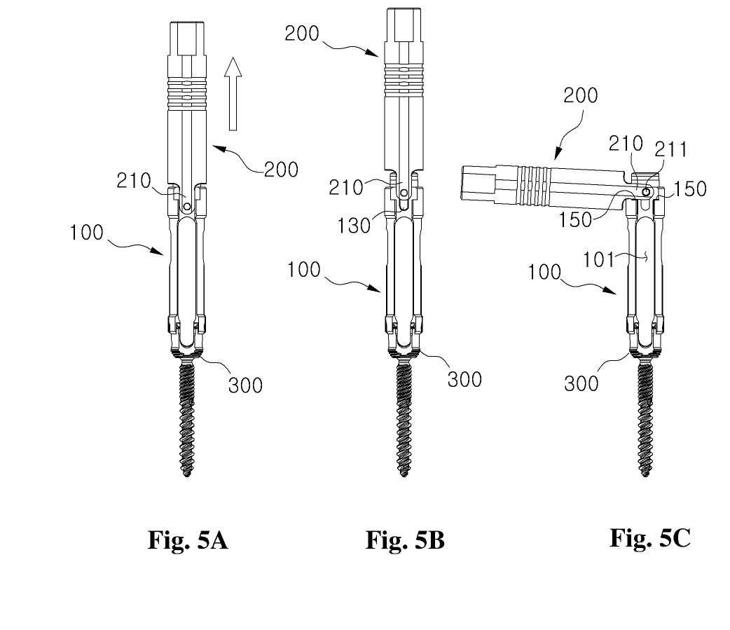

Also, FIGS. 5A-5C are views sequentially illustrating a procedure using the spinal screw holder for the minimal invasive surgery according to an embodiment of the present invention, wherein FIG. 5A is a conceptual view illustrating a state in which the first joint part and the second joint part, which are disposed in a straight line, are inserted, FIG. 5B is a conceptual view illustrating a state in which the second joint part is pulled in a direction that is away from an upper end of the first joint part, and FIG. 5C is a conceptual view illustrating a state in which the second joint part rotates to be bent with respect to the upper end of the first joint part.

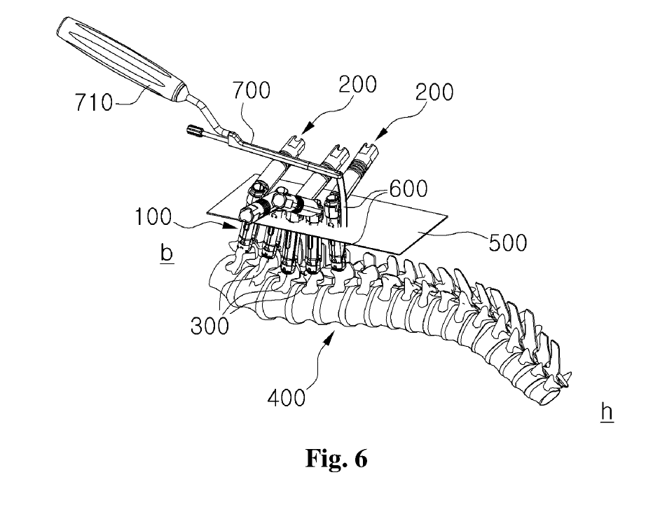

Also, FIGS. 6 to 8 are perspective views sequentially illustrating a procedure in which a rod is coupled in a state in which a spinal screw holder for a minimal invasive surgery is inserted through a skin of a subject according to an embodiment or another embodiment of the present invention.

For reference, non-explained reference numeral 350 in FIG. 1 denotes a spinal screw fitted to be fixed to a vertebra 400, and in FIGS. 6 to 8, reference symbol ‘h’ denotes a head direction of a subject, and reference symbol ‘b’ denotes a lower body direction of the subject.

As illustrated in the drawings, it is seen that the present invention has a structure in which a second joint part 200 is disposed in a straight line or rotatably bent with respect to a first joint part 100.

First, the first joint part 100 is a member having both penetrated ends, which is detachably coupled to a screw head 300 fixed to a vertebra (see reference numeral 400 of FIGS. 6 to 8) and exposed to a predetermined height through an opening (not shown) formed in a skin (see reference numeral 500 of FIGS. 6 to 8) of a subject.

A second joint part 200 is a member having both penetrated ends, which is coupled to an upper end of the first joint part 100, linearly movable in a direction that is away from the upper end of the first joint part 100 after being inserted through the opening in the state of being disposed in the straight line together with the first joint part 100, and rotates to be bent with respect to the upper end of the first joint part 100.

The foregoing embodiment as well as following various embodiments may be applied to the present invention.

First, the upper end of the first joint part 100 may have an outer diameter or width that is equal to or greater than an inner diameter or width of the second joint part 200 to prevent the second joint part 200 from being shaken or separated when the second joint part 200 is inserted through the opening in the state of being disposed in the straight line together with the first joint part 100.

Also, the bending of the second joint part 200 at an angle of about 90 degrees or less in both left and right directions with respect to the upper end of the first joint part 100 may be restricted after linearly moving in the direction that is away from the upper end of the first joint part 100 in the state of being disposed in the straight line together with the first joint part 100.

This is done because, when a rod (see reference numeral 600 of FIGS. 6 to 8) is inserted, if there is an interference-causing part on the skin 500, there is trouble in smooth insertion, and also, because a problem such as friction due to contact between the skin 500 of the subject and the second joint part 200 occurs when the second joint part 200 excessively rotates to be bent an angle of about 90 degrees or less.

The second joint part 200 may further include a plurality of slip prevention grooves 201 recessed along an outer surface of the second joint part 200 and vertically disposed in parallel to each other to prevent an entire components including the second joint part 200 from being damaged or separated due to slipping and dropping of the components from operator's hands.

In more detail, referring to FIGS. 2A-2C, the first joint part 100 may further include a first cutoff slit 110 having a predetermined width and cut by a predetermined length downward from an edge of the upper end of the first joint part 100 and a second cutoff slit 120 which has a predetermined width and is cut by a predetermined length downward from the edge of the upper end of the first joint part 100 and which is disposed in parallel to face the first cutoff slit 110.

Here, each of both left and right sides of the upper end of the first joint part 100 may have a diameter or width that is variable within a predetermined range with respect to the first cutoff slit 110 and the second cutoff slit 120.

Here, prevention holes 111 and 121 penetrated with a diameter greater than that of each of the first and second cutoff slits 110 and 120 may be further provided in lower ends of the first and second cutoff slits 110 and 120, respectively.

The prevention holes 111 and 121 are provided for the purpose of preventing deformation and damage such as cracks due to brittleness that may occur from the periphery of the lower ends of the first and second cutoff slits 110 and 120.

The present invention may further include a rotation-allowable guide groove 130 vertically recessed by a predetermined length in each of both sides of an upper portion of an outer surface of the first joint part 100, rotation pieces 210 as illustrated in FIGS. 3A-3C, and rotation pins (see reference numeral 202 FIG. 4) so that the second joint part 200 is rotatable with respect to the rotation-allowable guide groove 130.

The rotation pieces 210 respectively extend from both sides of an edge of the lower end of the second joint part 200 to face each other. The rotation pins 202 are inserted and fixed from through-holes 211 passing through the rotation pieces 210 and accommodate in the rotation-allowable guide groove 130, respectively.

Here, a lower end of the rotation-allowable guide groove 130 may be formed up to a position at which the edge of the lower end of the second joint part 200 is engaged with the edge of the upper end of the first joint part 100 so that the outer surfaces of the first joint part 100 and the second joint part 200 are disposed in the same line when the second joint part 200 is inserted through the opening in the state of being disposed in the straight line together with the first joint part 100 as illustrated in FIG. 5A.

Here, an upper end of the rotation-allowable guide groove 130 may be formed up to a position at which the second joint part 200 is bendable to be perpendicular to the first joint part 100 as illustrated in FIG. 5C by rotating to be bent after the second joint part 200 maximally linearly moves in a direction that is away from the upper end of the first joint part 100 as illustrated in FIG. 5B.

That is, each of both upper and lower ends of the rotation-allowable guide groove 130 may have a shape corresponding to the outer surface of each of the rotation pins 202 so that the second joint part 200 is not shaken when the second joint part 200 is inserted through the opening in the state of being disposed in the straight line together with the first joint part 100, and also, the second joint part 200 rotates to be bent after linearly moving in the direction that is away from the upper end of the first joint part 100.

Furthermore, the present invention may further include a rotation-allowable cutoff surface 140 and an excessive rotation restriction protrusion 150 so that the second joint part 200 rotates to be bent with respect to the upper end of the first joint part 100.

The rotation-allowable cutoff surface 140 may be cut in a flat shape from the edge of the upper end of the first joint part 100 and formed on each of both outer surfaces of the upper end of the first joint part 100.

The excessive rotation restriction protrusion 150 may protrude to be stepped on each of both sides of the edge of the lower end of the rotation-allowable cutoff surface 140. When the second joint part 200 rotates with respect to the upper end of the first joint part 100, an edge of one side of each of the rotation pieces 210 extending from both sides of the edge of the lower end of the second joint part 200 contacts the excessive rotation restriction protrusion 150, and thus, the second joint part 200 is perpendicular to the first joint part 100.

Furthermore, it is seen that the upper end of the rotation-allowable guide groove 130 is formed up to a position at which the rotation pieces 210 are perpendicular to the rotation-allowable guide groove 130.

In more detail, referring to FIGS. 2A and 4, the first joint part 100 may further include rod insertion guide cutoff parts 101 and a clamp 160 so that the screw head 300 is smoothly coupled and separated, and also, a rod 600 is accurately inserted and seated in rod insertion grooves 301.

First, each of the rod insertion guide cutoff parts 101 are cut to a predetermined height upward from each of both sides of the edge of the lower end of the first joint part 100 to communicate with each of the rod insertion grooves 301 that are cut downward from both sides of the edge of the upper end of the screw head 300.

The clamp 160 is disposed on both sides of the first joint part 100 so as to be perpendicular to a virtual line passing through a center of each of the rod insertion guide cutoff parts 101 and has an upper end fixed to each of both side surfaces of the first joint part 100 and a lower end that is elastically deformable with respect to each of both side surfaces of the first joint part 100.

Here, both side surfaces of the screw head 300 may be engaged with and fixed to lower ends of facing inner surfaces of the clamp 160 and rotate forwardly and reversely with respect to the screw head 300.

Here, the forward and reverse rotatable structure of the first joint part 100 with respect to the screw head 300 may be called a coupling structure that is provided to align and guide a position of the rod when the rod is inserted in order as illustrated in FIGS. 6 to 8 to simultaneously fix the plurality of screw heads 300 fixed when the plurality of screw heads 300 are fixed along a plurality of vertebrae 400.

Also, the clamp 160 will be described in more detail with reference to FIGS. 2A-2C and 4.

First, the clamp 160 may include a deformation-allowable bar 161 having a linear bar shape and disposed between a pair of cutoff slits 103 that is linearly cut from both side surfaces of the first joint part 10 to the lower end of the first joint part 100.

Also, the clamp 160 may include a clamping piece 162 which extends from a lower end of the deformation-allowable bar 161 to form a predetermined shape and of which an edge of a lower end is disposed in the same line as the edge of the lower end of the first joint part 100.

Also, the clamp 160 may include a clamping rib 163 protruding from each of both sides of facing inner surfaces of the clamp pieces 162 in a direction in which the clamping pieces 162 face each other and engaged with a clamping groove 303 recessed from an outer surface of the screw head 300.

Here, the virtual line passing through a center of each of the clamping pieces 162 may be perpendicular to a virtual line passing through each of the rod insertion guide cutoff parts 101.

Thus, when the fixed state of the clamping piece 162 engaged with the screw head 300 is released, and the first joint part 100 rotates at an angle of 90 degrees, the first joint part 100 may be separated from the screw head 300.

Also, the clamp 160 may further include a deformation-allowable groove 164 recessed at a predetermined thickness from each of both side surfaces of the first joint part 100 and having a predetermined area on an area including the upper end of the deformable-allowable bar 161.

The deformation-allowable groove 164 may be called a technical unit that allows a thickness of each of both side surfaces of the first joint part 100 to be thinner so that the deformation-allowable bar 161 is more smoothly elastically deformed from both side surfaces of the first joint part 100 together with the clamping pieces 162.

The first joint part 100 may further include a clamping extension rib 170 protruding from an edge of each of both sides of the lower end of each of the rod insertion guide cutoff parts 101 along the inner side surface of the first joint part 100 and disposed on the same circle as the clamping rib 163.

Here, the clamping extension rib 170 may be provided to provide a portion that is engaged and fixed over the entire clamping groove 303 of the screw head 300 together with the clamping rib 163.

Here, a first clamping guide slant surface 163s may be formed by being cut and removed to be inclined upward from a bottom surface 163 of the clamping rib 163, and a second clamping guide slant surface 170s may be formed by being cut and removed to be inclined upward from a bottom surface of the clamping extension rib 170. This may be grasped through the enlarged view of the left side in FIG. 4.

The first clamping guide slant surface 163s and the second clamping guide slant surface 170s may be provided to guide the clamping rib 163 and the clamping extension rib 170 so that the clamping rib 163 and the clamping extension rib 170 are smoothly engaged to be inserted into the clamping grooves 303 of the screw head 300.

It is seen that the first joint part 100 further includes head hook ribs 180 protruding from lower portions of edges of both sides of the rod insertion guide cutoff parts 101 in directions facing each other as shown in the enlarged view of the left side of FIG. 4, and edges of lower ends of the head hook ribs 180 are parallel to the edge of the lower end of the first joint part 100.

Here, the head hook ribs 180 may prevent the edge of the upper end of the screw head 300 from further ascending or prevent the first joint part 100 from further descending toward the screw head 300 and also may function as a position determination unit for determining a hooked and fixed position of the upper end of the screw head 300 with respect to the first joint part 100.

Thus, when the surgery is completed, the operator pushes a clamping release tool (not shown) from the upper end of the second joint part 200 to separate the inner side surfaces of the clamp 160 including the clamping pieces 162 engaged with both side surfaces of the screw head 300 from each other in a direction in which the inner side surfaces are away from each other and then allow the first joint part 100 to rotate at an angle of about 90 degrees with respect to the screw head 300.

Here, the clamping pieces 162 may be respectively disposed in the rod insertion grooves 301 of the screw head 300 to release the engagement of the clamping rib 163 and the clamping extension rib 170 with respect to the clamping groove 303, thereby separating the first joint part 100 from the screw head 300.

Hereinafter, a procedure using the spinal screw holder for the minimal invasive surgery according to an embodiment of the present invention will be briefly described with reference to FIGS. 6 to 8.

First, the insertion of the rod is performed by holding a handle 710 disposed on one side of a rod holder 700 rotatably coupled to one end of the rod 600 having an arc shape corresponding to a lordotic curve, i.e., a lordosis as illustrated in FIG. 6 and pushing the rod 60 between the skin 100 of the subject and the rod insertion guide cutoff parts 101 of the first joint part 100.

Here, whenever an end of the rod 600 is intended to pass through the rod insertion guide cutoff parts 101 of each of the plurality of first joint parts 100, the operator may adequately rotate the first joint part 100 with respect to the screw head 300 to align the positions of the rod insertion guide cutoff parts 101.

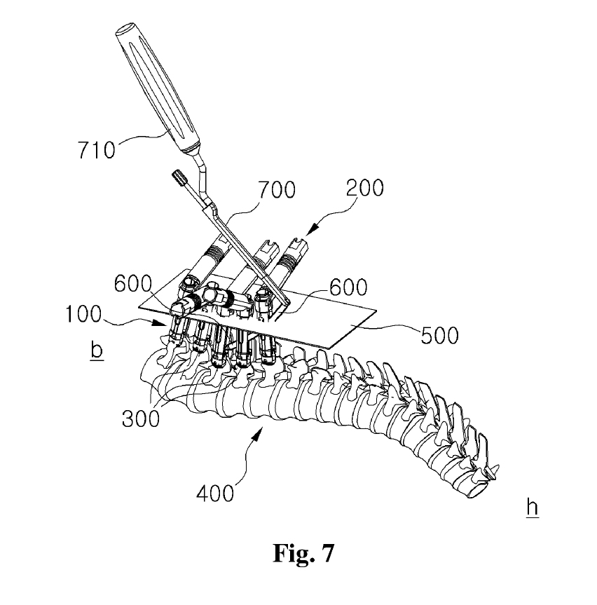

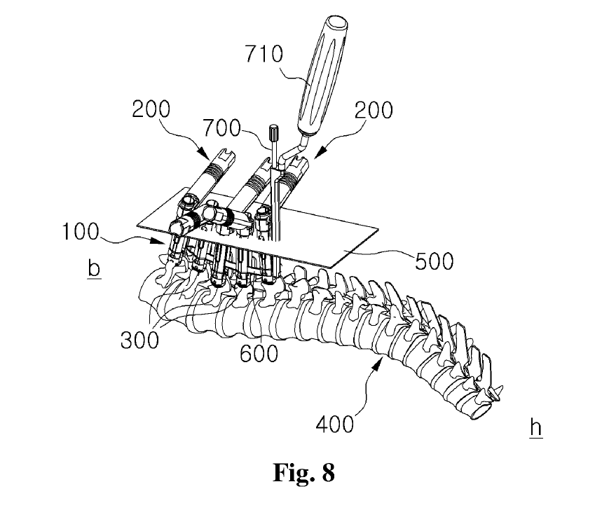

Thereafter, the operator may push the rod 600 and the rod holder 700 through the skin 500 of the subject as illustrated in FIGS. 7 and 8 to maintain the state in which the rod 600 is inserted to pass through all of the rod insertion guide cutoff parts 101 of the plurality of first joint parts and then to proceed to the next procedure.

### Second Embodiment

The same reference numerals are used for the same components as those of the first embodiment described above, and important contents in the second embodiment will be mainly described.

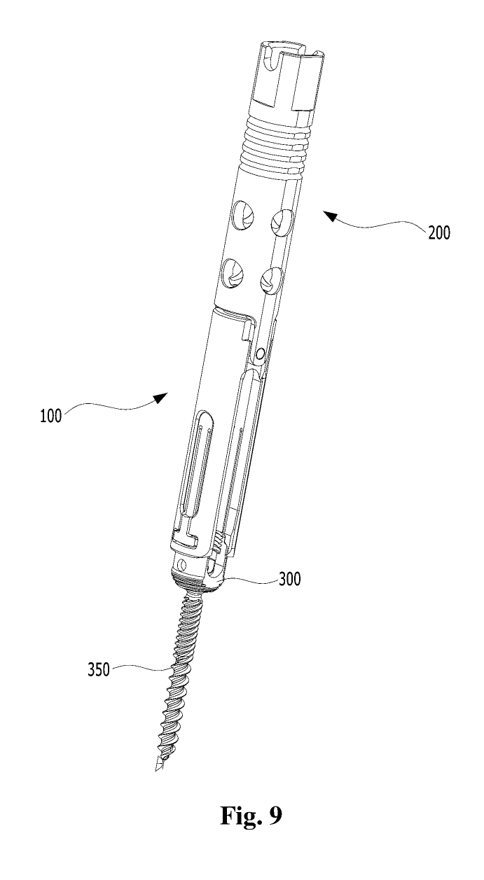

First, FIG. 9 is a perspective view illustrating a state in which the spinal screw holder for the minimal invasive surgery is coupled to a screw head according to another embodiment of the present invention.

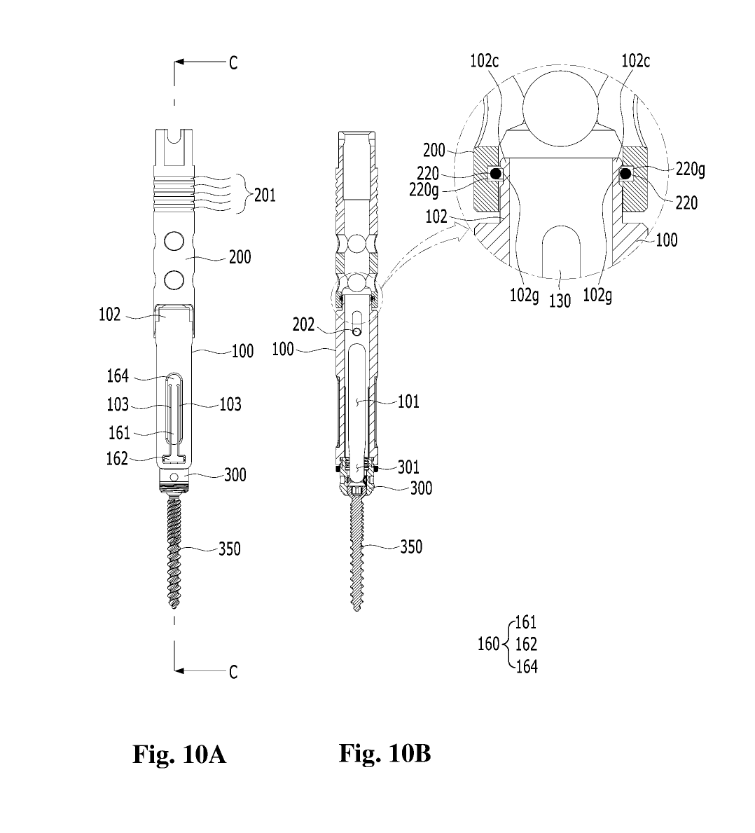

Also, FIGS. 10A and 10B are views illustrating a state in which the spinal screw holder for the minimal invasive surgery is coupled to the screw head according to another embodiment of the present invention, wherein FIG. 10A is a front view, and FIG. 10B is a cross-sectional view.

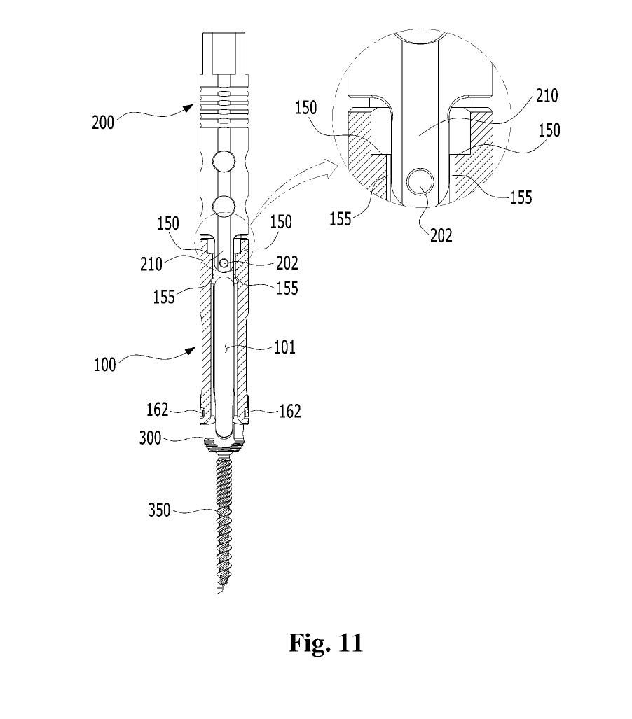

Also, FIG. 11 is a side view illustrating a state in which the spinal screw holder for the minimal invasive surgery is coupled to the screw head according to another embodiment of the present invention.

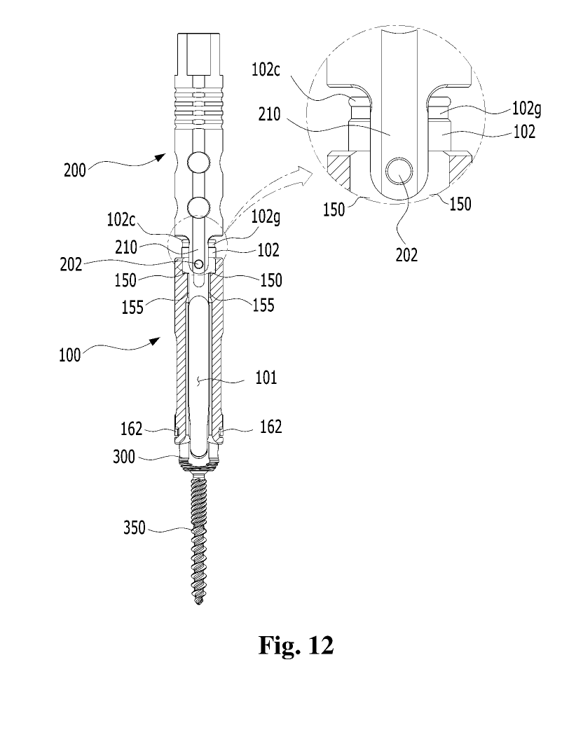

Also, FIG. 12 is a conceptual view illustrating a state in which a second joint part constituting the spinal screw holder for the minimal invasive surgery moves in a direction that is away from the first joint part to separate a lower end of the second joint part from an upper end of the first joint part in FIG. 11.

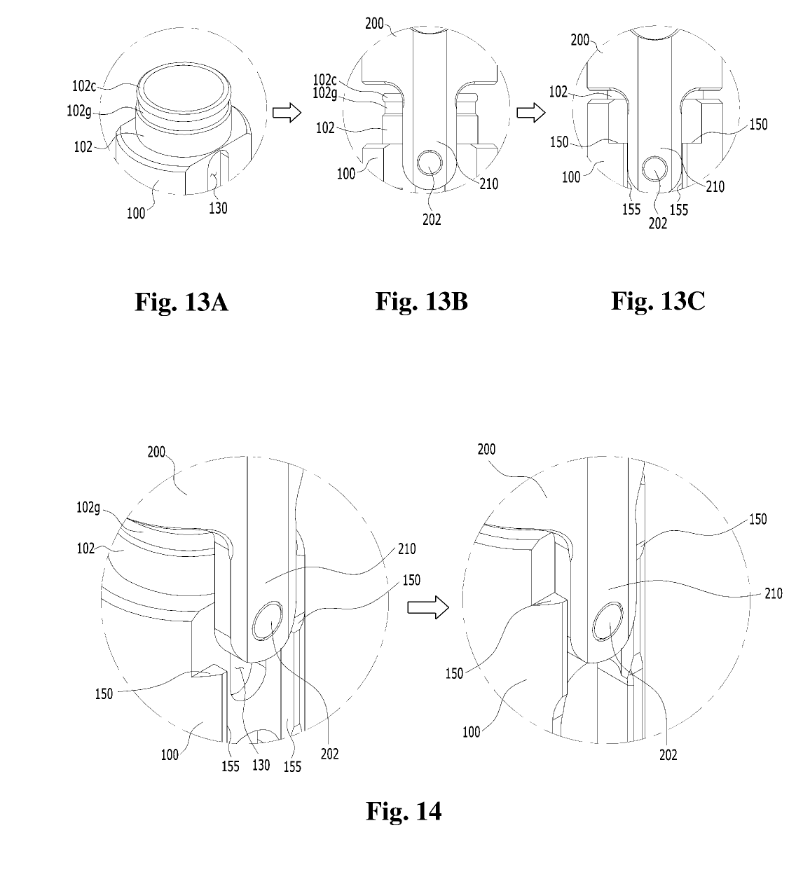

Also, FIGS. 13A-13C are partial enlarged views illustrating a main part of the spinal screw holder for the minimal invasive surgery according to another embodiment of the present invention, wherein FIG. 13A is a view illustrating an upper end of the first joint part, FIG. 13B is a view illustrating a state in which the second joint part is coupled to the first joint part, and FIG. 13C is a partial enlarged view illustrating a state in which the second joint part and the second joint part are coupled to each other.

Also, FIG. 14 is a partial enlarged view illustrating a main part of the spinal screw holder for the minimal invasive surgery according to another embodiment of the present invention, wherein the first view illustrates a state in which the second joint part is coupled to the first joint part when viewed at an oblique angle, and the second view illustrates a state in which the second joint part is coupled to the first joint part when viewed at an oblique angle.

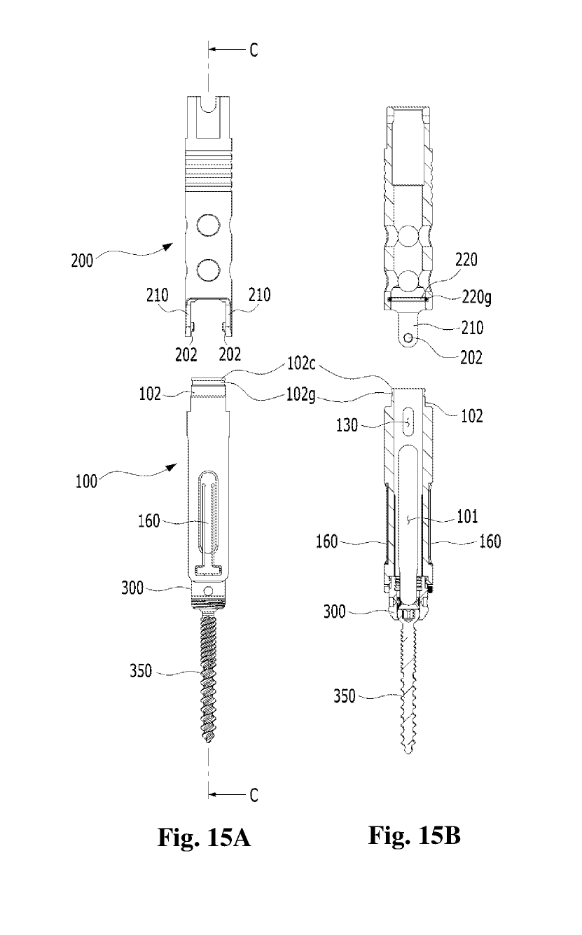

Also, FIGS. 15A and 15B are views illustrating a state in which the first joint part and the second joint part, which constitute the spinal screw holder for the minimal invasive surgery, are separated from each other, wherein FIG. 15A is a front view, and FIG. 15B is a cross-sectional view.

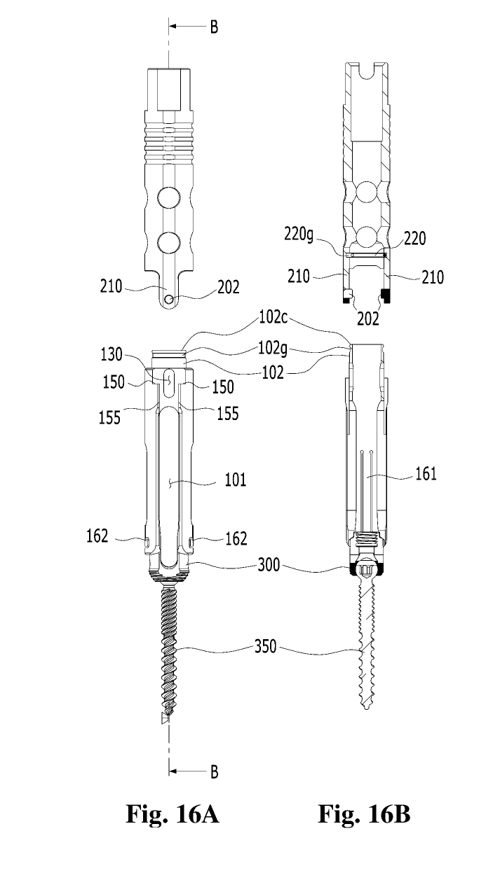

Also, FIGS. 16A and 16B are views illustrating a state in which the first joint part and the second joint part, which constitute the spinal screw holder for the minimal invasive surgery, are separated from each other, wherein FIG. 16A is a side view, and FIG. 16B is a cross-sectional view.

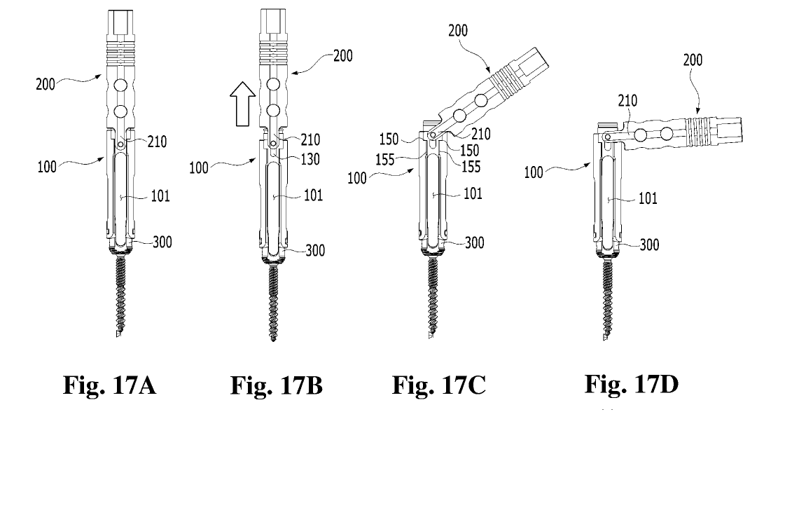

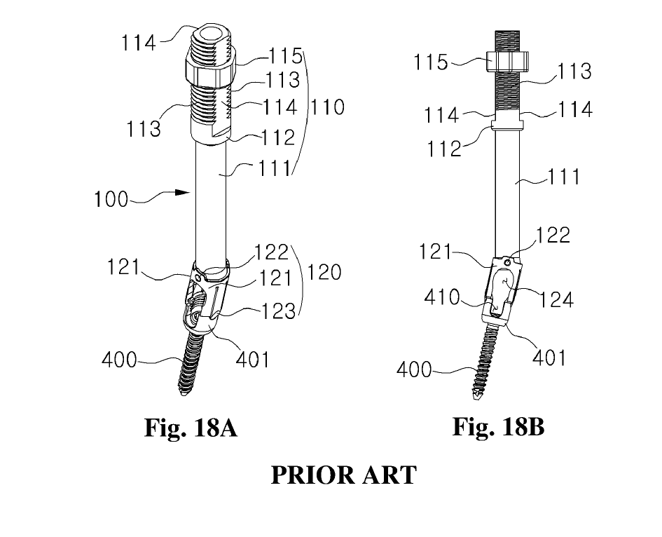

Also, FIGS. 17A-17D are views sequentially illustrating a procedure using the spinal screw holder for the minimal invasive surgery according to another embodiment of the present invention, wherein FIG. 17A is a conceptual view illustrating a state in which the first joint part and the second joint part, which are disposed in a straight line, are inserted, FIG. 17B is a conceptual view illustrating a state in which the second joint part is pulled in a direction that is away from an upper end of the first joint part, and FIG. 17C is a conceptual view illustrating a state in which the second joint part rotates to be bent at a predetermined angle with respect to the upper end of the first joint part, and FIG. 17D is a conceptual view illustrating a state in which the second joint part completely rotates to be bent with respect to the upper end of the first joint part.

As illustrated in the drawings, it is seen that the present invention has a structure in which a second joint part 200 is disposed in a straight line or rotatably bent with respect to a first joint part 100.

Like the first embodiment, when the second embodiment is described with reference to FIGS. 6 to 8, the first joint part 100 is a member having both penetrated ends, which is detachably coupled to a screw head 300 fixed to a vertebra 400 and exposed to a predetermined height through an opening (not shown) formed in a skin 500 of a subject.

The second joint part 200 is a member having both penetrated ends, which is coupled to an upper end 102 of the first joint part 100, linearly movable in a direction that is away from the upper end 102 of the first joint part 100 after being inserted through the opening in the state of being disposed in the straight line together with the first joint part 100, and rotates to be bent with respect to the upper end 102 of the first joint part 100.

The upper end 102 of the first joint part 100 may have a diameter less than that of a main body of the first joint part 100 and also have a predetermined length. Thus, the lower end of the second joint part 200 linearly moves without being shaken when moving downward to be coupled while accommodating the upper end 102 of the first joint part 100 therein.

For example, the upper end 102 may have a diameter less about 4 mm to about 5 mm than that of the main body of the first joint part 100 and a length of about 5 mm to about 7 mm.

The upper end 102 may have an outer diameter that is equal to or slightly less than that of a lower end of the second joint part 200 so as to be smoothly coupled. Since the lower end of the second joint part 200 moves while accommodating the protruding upper end 102 therein, the lower end may linearly move to be coupled without being shaken.

The bending of the second joint part 200 at an angle of 90 degrees or less in both left and right directions with respect to the upper end 102 of the first joint part 100 may be restricted after linearly moving in the direction that is away from the upper end 102 of the first joint part 100 in the state of being disposed in the straight line together with the first joint part 100.

This is done because, when a rod (see reference numeral 600 of FIGS. 6 to 8) is inserted, if there is an interference-causing part on the skin 500, there is trouble in smooth insertion, and also, because a problem such as friction due to contact between the skin 500 of the subject and the second joint part 200 occurs when the second joint part 200 excessively rotates to be bent an angle of about 90 degrees or less.

The second joint part 200 may further include a plurality of slip prevention grooves 201 recessed along an outer surface of the second joint part 200 and vertically disposed in parallel to each other to prevent an entire components including the second joint part 200 from being damaged or separated due to slipping and dropping of the components from operator's hands.

The upper end 102 of the first joint part 100 may have an outer diameter or width that is equal to or greater than an inner diameter or width of the second joint part 200 to prevent the second joint part 200 from being shaken or separated when the second joint part 200 is inserted through the opening in the state of being disposed in the straight line together with the first joint part 100.

Here, although not shown in FIGS. 9 to 17D, as illustrated in FIGS. 2A-2C, the first joint part 100 may further include a first cutoff slit 110 having a predetermined width and cut by a predetermined length downward from an edge of the upper end of the first joint part 100 and a second cutoff slit 120 which has a predetermined width and is cut by a predetermined length downward from the edge of the upper end of the first joint part 100 and which is disposed in parallel to face the first cutoff slit 110.

Here, each of both left and right sides of the upper end of the first joint part may have a diameter or width that is variable within a predetermined range with respect to the first cutoff slit 110 and the second cutoff slit 120.

Here, prevention holes 111 and 121 penetrated with a diameter greater than that of each of the first and second cutoff slits 110 and 120 may be further provided in lower ends of the first and second cutoff slits 110 and 120, respectively.

The prevention holes 111 and 121 are provided for the purpose of preventing deformation and damage such as cracks due to brittleness that may occur from the periphery of the lower ends of the first and second cutoff slits 110 and 120.

Referring to FIGS. 13A to 16B, the present invention may further include a rotation-allowable guide groove 130 vertically recessed by a predetermined length in each of both sides of an upper portion of an outer surface of the first joint part 100, rotation pieces 210, rotation pins 202, and a linear movement guide protrusion 155 so that the second joint part 200 is rotatable with respect to the rotation-allowable guide groove 130.

The rotation pieces 210 extend from both sides of an edge of the lower end of the second joint part 200 to face each other. The rotation pins 202 are inserted and fixed from through-holes 211 passing through the rotation pieces 210 and accommodate in the rotation-allowable guide groove 130, respectively.

Here, a lower end of the rotation-allowable guide groove 130 may be formed up to a position at which the edge of the lower end of the second joint part 200 is engaged with the edge of the upper end of the first joint part 100 so that the outer surfaces of the first joint part 100 and the second joint part 200 are disposed in the same line when the second joint part 200 is inserted through the opening in the state of being disposed in the straight line together with the first joint part 100 as illustrated in FIG. 17A.

Here, an upper end of the rotation-allowable guide groove 130 may be formed up to a position at which the second joint part 200 is bendable to be perpendicular to the first joint part 100 as illustrated in FIG. 17D by rotating to be bent after the second joint part 200 maximally linearly moves in a direction that is away from the upper end of the first joint part 100 as illustrated in FIG. 17B.

That is, each of both upper and lower ends of the rotation-allowable guide groove 130 may have a shape corresponding to the outer surface of each of the rotation pins 202 so that the second joint part 200 is not shaken when the second joint part 200 is inserted through the opening in the state of being disposed in the straight line together with the first joint part 100, and also, the second joint part 200 rotates to be bent after linearly moving in the direction that is away from the upper end of the first joint part 100.

Referring to FIGS. 11 to 14, the linear movement guide protrusion 155 is disposed in a longitudinal direction of the main body of the first joint part 100 on both sides of the rotation pieces 210 so that the rotation pieces 210 do not rotate but linearly moves when the second joint part 200 moves downward to be coupled.

Particularly, the linear movement guide protrusion 155 may be disposed on each of both sides with respect to one rotation-allowable guide groove 130 on an upper portion of the main body of the first joint part 100, and each of the linear movement guide protrusions 155 is disposed perpendicular to an excessive rotation restriction protrusion 150.

A distance between the linear movement guide protrusions 155 that are spaced apart from each other with respect to the rotation-allowable guide groove 130 may be equal to or slightly greater than a width of each of the rotation pieces 210. Also, the linear movement guide protrusion 155 may protrude to be stepped on each of both sides of the rotation-allowable guide groove 130.

Due to the above-described structure, when the second joint part 200 moves to the first joint part 100 to be coupled, the rotation pieces 210 may be inserted between the linear movement guide protrusions 155 to move without being shaken in a left and right direction.

As the rotation pieces 210 are linearly movable without being shaken, the second joint part 200 extending from the upper portion of each of the rotation pieces 210 may also linearly move without being shaken.

Referring to FIGS. 10A and 13C, an upper end groove 102g having a predetermined width and depth may be formed in an upper circumference of the upper end 102 of the first joint part 100, and a groove coupling part 220 fitted into the upper end groove 102g to fix the first joint part 100 to the second joint part 200 when coupled to the upper end 102 of the first joint part 100 is provided in an inner circumference of the lower end of the second joint part 200.

In more particularly, the upper end groove 102g having a width of about 1 mm to about 1.5 mm and a depth of about 0.4 mm to about 0.5 mm may be formed to be recessed from the upper circumference of the upper end 102 of the first joint part 100. An upper end protrusion 102c convexly extending to correspond to the diameter of the upper end 102 is disposed on the upper circumference of the upper end groove 102g.

A lower end groove 220g having a width of about 1 mm and a depth of about 0.6 mm to about 0.8 mm is formed in the inner circumference of the lower end of the second joint part 200, and the groove coupling part 220 is fixed and installed in a ring shape in the lower end groove 220g. The ring 200 is coupled to in a state of slightly protruding from the lower end groove 220g.

When the second joint part 200 is coupled to the upper end 102 of the first joint part 100, the ring 220 may be fitted into the upper end groove 102g, and the upper end protrusion 102c may be hung on the ring 220 to stably fix the first joint part 100 and the second joint part 200. Due to this coupling method, the operator may surely know that the first joint part 100 and the second joint part 200 are coupled to each other to improve surgical convenience.

Referring to FIGS. 12 to 14, the present invention may further include a rotation-allowable cutoff surface 140 and an excessive rotation restriction protrusion 150 so that the second joint part 200 rotates to be bent with respect to the upper end of the first joint part 100.

The rotation-allowable cutoff surface 140 may be cut in a flat shape from the edge of the upper end of the first joint part 100 and formed on each of both outer surfaces of the upper end of the first joint part 100.

The excessive rotation restriction protrusion 150 may protrude to be stepped on each of both sides of the edge of the lower end of the rotation-allowable cutoff surface 140. When the second joint part 200 rotates with respect to the upper end of the first joint part 100, an edge of one side of each of the rotation pieces 210 extending from both sides of the edge of the lower end of the second joint part 200 contacts the excessive rotation restriction protrusion 150, and thus, the second joint part 200 is perpendicular to the first joint part 100.

Furthermore, it is seen that the upper end of the rotation-allowable guide groove 130 is formed up to a position at which the rotation pieces 210 are perpendicular to the rotation-allowable guide groove 130.

In more detail, referring to FIGS. 10A and 10B, the first joint part 100 may further include rod insertion guide cutoff parts 101 and a clamp 160 so that the screw head 300 is smoothly coupled and separated, and also, a rod 600 is accurately inserted and seated in rod insertion grooves 301.

First, each of the rod insertion guide cutoff parts 101 are cut to a predetermined height upward from each of both sides of the edge of the lower end of the first joint part 100 to communicate with each of the rod insertion grooves 301 that are cut downward from both sides of the edge of the upper end of the screw head 300.

The clamp 160 is disposed on both sides of the first joint part 100 so as to be perpendicular to a virtual line passing through a center of each of the rod insertion guide cutoff parts 101 and has an upper end fixed to each of both side surfaces of the first joint part 100 and a lower end that is elastically deformable with respect to each of both side surfaces of the first joint part 100.

Here, both surfaces of the screw head 300 may be engaged with and fixed to lower ends of facing inner surfaces of the clamp 160 and rotate forwardly and reversely with respect to the screw head 300.

Here, the forward and reverse rotatable structure of the first joint part 100 with respect to the screw head 300 may be called a coupling structure that is provided to align and guide a position of the rod when the rod is inserted in order as illustrated in FIGS. 6 to 8 to simultaneously fix the plurality of screw heads 300 fixed when the plurality of screw heads 300 are fixed along a plurality of vertebrae 400.

Also, the clamp 160 will be described again in more detail with reference to FIGS. 10A and 10B.

First, the clamp 160 may include a deformation-allowable bar 161 having a linear bar shape and disposed between a pair of cutoff slits 103 that is linearly cut from both side surfaces of the first joint part 10 to the lower end of the first joint part 100.

Also, the clamp 160 may include a clamping piece 162 which extends from a lower end of the deformation-allowable bar 161 to form a predetermined shape and of which an edge of a lower end is disposed on the lower end of the first joint part 100.

Also, the clamp 160 may include a clamping rib 163 protruding from each of both sides of facing inner surfaces of the clamp pieces 162 in a direction in which the clamping pieces 162 face each other and engaged with a clamping groove 303 recessed from an outer surface of the screw head 300.

Here, the virtual line passing through a center of each of the clamping pieces 162 may be perpendicular to a virtual line passing through each of the rod insertion guide cutoff parts 101.

Thus, when the fixed state of the clamping piece 162 engaged with the screw head 300 is released, and the first joint part 100 rotates at an angle of 90 degrees, the first joint part 100 may be separated from the screw head 300.

Also, the clamp 160 may further include a deformation-allowable groove 164 recessed at a predetermined thickness from each of both side surfaces of the first joint part 100 and having a predetermined area on an area including the upper end of the deformable-allowable bar 161.

The deformation-allowable groove 164 may be called a technical unit that allows a thickness of each of both side surfaces of the first joint part 100 to be thinner so that the deformation-allowable bar 161 is more smoothly elastically deformed from both side surfaces of the first joint part 100 together with the clamping pieces 162.

When the surgery is completed, the operator pushes a clamping release tool (not shown) from the upper end of the second joint part 200 to separate the inner side surfaces of the clamp 160 including the clamping pieces 162 engaged with both side surfaces of the screw head 300 from each other in a direction in which the inner side surfaces are away from each other and then allow the first joint part 100 to rotate at an angle of about 90 degrees with respect to the screw head 300, thereby releasing the engagement so that the first joint part 100 is separated from the screw head 300.

The procedure using a spinal screw holder for minimal invasive surgery according to another embodiment of the present invention has been described in detail in the first embodiment with reference to FIGS. 6 to 8, and thus, its detailed description will be omitted.

Hereinafter, an effect of the spinal screw holder for the minimal invasive surgery according to an embodiment of the present invention will be described as follows.

First, the present invention may include the first joint part having both penetrated ends, which is detachably coupled to the screw head fixed to the vertebra so as to be exposed to a predetermined height through the opening formed in the skin of the subject and the second joint part having both penetrated ends, which is coupled to the upper end of the first joint part and which is linearly movable in the direction that is away from the upper end of the first joint part and rotates to be bent with respect to the upper end of the first joint part after the second joint part is inserted through the opening in the state of being disposed in a straight line together with the first joint part. Thus, the interference of the fixing rod may be maximally avoided during the surgery so that the fixing rod is smoothly inserted to be fixed to the screw head, thereby improving surgical convenience.

Also, according to the present invention, the second joint part may linearly move without being shaken when the lower end of the second joint part, which is disposed at the relatively upper side, of the two joint parts constituting the spinal screw holder is fitted to be coupled to the upper end of the first joint part disposed at the relatively lower side so that the surgery is quickly and stably performed when the lordosis of the vertebra is formed by closely attaching the spinal screw holders to each other after the two joint parts are coupled to each other by the operator.

Also, according to the present invention, the linear movement guide protrusion disposed on each of both sides of the rotation piece in the longitudinal direction of the main body of the first joint part so that the rotation pieces disposed on both edges of the lower end of the second joint part to face each other linearly moves without rotating when the second joint part that is disposed at relatively upper side moves downward to be coupled may be provided so that the second joint part linearly and stably moves without being shaken when the second joint part is fitted to be coupled to the upper end of the upper end of the first joint part.

Also, according to the present invention, the upper end groove having a predetermined width and depth may be formed in the upper circumference of the upper end of the first joint part, and the lower end groove having a predetermined width and depth may be formed in the inner circumference of the lower end of the second joint part. Thus, when the ring is fixed and installed in the lower end groove to couple the second joint part to the upper end of the first joint part, the ring may be coupled to the upper end groove while being clacked. Therefore, the operator may surely know that the first joint part and the second joint part are coupled to each other to improve the safety of the patient as well as the surgical convenience.

Furthermore, the present invention may further include the first cutoff slit having a predetermined width and cut by a predetermined length downward from the edge of the upper end of the first joint part and a second cutoff slit which has a predetermined width and is cut by a predetermined length downward from the edge of the upper end of the first joint part and which is disposed in parallel to face the first cutoff slit. Here, each of both left and right sides of the upper end of the first joint part may have the diameter or width that is variable within a predetermined range with respect to the first cutoff slit and the second cutoff slit, and the upper end of the first joint part may have the outer diameter or width that is equal to or greater than the inner diameter or width of the lower end of the second joint part. Thus, when the lower end of the second joint part and the upper end of the first joint part are coupled to each other, the gap between the two cutoff slits may be narrowed so that the second joint part is coupled without being shaken, thereby providing the surgical convenience and the safety. Also, the coupled state of the first joint part and the second joint part may be maintained without being separated or shaken to allow the operation to adjust the position of the second joint part by holding the second joint part, thereby sufficiently securing the area to be grasped.

As described above, the basic technical idea of the present invention is to provide the spinal screw holder for the minimal invasive surgery, which allows the fixing rod to be smoothly inserted into and fixed to the screw head while maximally avoiding the interference in the procedure.

According to the present invention having the above-described constitutions, the following effects may be attained.

First, the present invention may include: the first joint part having both penetrated ends, which is detachably coupled to the screw head fixed to the vertebra so as to be exposed to a predetermined height through the opening formed in the skin of the subject; and the second joint part having both penetrated ends, which is coupled to an upper end of the first joint part and which is linearly movable in the direction that is away from the upper end of the first joint part, and rotates to be bent with respect to the upper end of the first joint part after the second joint part is inserted through the opening in the state of being disposed in a straight line together with the first joint part. Thus, the interference of the fixing rod may be maximally avoided during the surgery so that the fixing rod is smoothly inserted to be fixed to the screw head, thereby improving surgical convenience.

That is to say, according to the present invention, the second joint part may rotate to be bent with respect to the upper end of the first joint part so that there is no part to cause the interference on the skin of the subject during the surgery in which the rod is inserted through the opening of the skin so as to be fixed to the screw head fixed to the vertebra. Thus, the rod may be easily inserted.

Also, the upper end of the first joint according to the present invention may have the outer diameter or width that is equal to or greater than the inner diameter or width of the second joint part. Thus, as described above, when the second joint part is inserted through the opening in the state of being disposed in the straight line together with the first joint part, the second joint part be coupled without being shaken to improve the surgical convenience and safety. In addition, the coupled state between the first joint part and the second joint part may be maintained without being separated or shaken to sufficiently secure the area to be grasped by the operator so that the operator holds the second joint part to adjust the position of the second joint part.

Also, according to the present invention, the bending of the second joint part at an angle of about 90 degrees or less in both left and right directions with respect to the upper end of the first joint part may be restricted after linearly moving in the direction that is away from the upper end of the first joint part in the state of being disposed in the straight line together with the first joint part. Thus, the position of the second joint part on the skin of the subject may be adjusted again so that there is no components causing the interference when the rod is inserted, and also, the problem such as the friction due to the contact between the skin of the subject and the second joint part when excessively rotating may be prevented from occurring.

Also, the present invention may further include the first cutoff slit having a predetermined width and cut by a predetermined length downward from the edge of the upper end of the first joint part and a second cutoff slit which has a predetermined width and is cut by a predetermined length downward from the edge of the upper end of the first joint part and which is disposed in parallel to face the first cutoff slit. Here, each of both left and right sides of the upper end of the first joint part may have the diameter or width that is variable within a predetermined range with respect to the first cutoff slit and the second cutoff slit, and the upper end of the first joint part may have the outer diameter or width that is equal to or greater than the inner diameter or width of the lower end of the second joint part. Thus, when the lower end of the second joint part and the upper end of the first joint part are coupled to each other, the gap between the two cutoff slits may be narrowed so that the second joint part is coupled without being shaken, thereby providing the surgical convenience and the safety.

Also, according to the present invention, rotation-allowable cutoff surface which is cut in a flat shape from the edge of the upper end of the first joint part and is formed on each of both outer surfaces of the upper end of the first joint part and an excessive rotation restriction protrusion protruding to be stepped on each of both sides of the edge of the lower end of the rotation-allowable cutoff surface and contacting the edge of one side of the rotation piece extending from each of both sides of the edge of the lower end of the second joint part when the second joint part rotates with respect to the upper end of the first joint part so as to allow the second joint part to be perpendicular to the first joint part may be further provided. Thus, the position of the second joint part on the skin of the subject may be adjusted again so that there is no components causing the interference when the rod is inserted, and also, the problem such as the friction due to the contact between the skin of the subject and the second joint part when excessively rotating may be prevented from occurring.

Also, according to the present invention, the second joint part may linearly move without being shaken when the lower end of the second joint part, which is disposed at the relatively upper side, of the two joint parts constituting the spinal screw holder is fitted to be coupled to the upper end of the first joint part disposed at the relatively lower side so that the surgery is quickly and stably performed when the lordosis of the vertebra is formed by closely attaching the spinal screw holders to each other after the two joint parts are coupled to each other by the operator.

Also, the linear movement guide protrusion disposed on each of both sides of the rotation piece in the longitudinal direction of the main body of the first joint part so that the rotation pieces disposed on both edges of the lower end of the second joint part to face each other linearly moves without being shaken when the second joint part that is disposed at relatively upper side moves downward to be coupled may be provided so that the second joint part linearly and stably moves without shaken when the second joint part is fitted to be coupled to the upper end of the upper end of the first joint part.

Also, the upper end groove having a predetermined width and depth may be formed in the upper circumference of the upper end of the first joint part, and the lower end groove having a predetermined width and depth may be formed in the inner circumference of the lower end of the second joint part. Thus, when the ring is fixed and installed in the lower end groove to couple the second joint part to the upper end of the first joint part, the ring may be coupled to the upper end groove while being clacked. Therefore, the operator may surely know that the first joint part and the second joint part are coupled to each other to improve the safety of the patient as well as the surgical convenience.

Also, it should be understood that numerous other modifications and embodiments can be devised by those skilled in the art that will fall within the scope of the fundamental technical idea of the principles of the present invention.

What is claimed is:

1. A spinal screw holder for a minimal invasive surgery, comprising:
a first joint part having both penetrated ends, which is detachably coupled to a screw head fixed to a vertebra so as to be exposed to a predetermined height through an opening formed in a skin of a subject; and
a second joint part having both penetrated ends, which is coupled to an upper end of the first joint part and which is linearly movable in a direction that is away from the upper end of the first joint part and rotates to be bent with respect to the upper end of the first joint part after the second joint part is inserted through the opening in a state of being disposed in a straight line together with the first joint part,
wherein the upper end of the first joint part has an outer diameter or width that is equal to or greater than an inner diameter or width of the lower end of the second joint part,
the first joint part further comprises:
a first cutoff slit having a predetermined width and cut by a predetermined length downward from an edge of the upper end of the first joint part; and
a second cutoff slit which has a predetermined width and is cut by a predetermined length downward from the edge of the upper end of the first joint part and which is disposed in parallel to face the first cutoff slit, and
each of both left and right sides of the upper end of the first joint part have a diameter or width that is variable within a predetermined range with respect to the first cutoff slit and the second cutoff slit.

2. A spinal screw holder for a minimal invasive surgery, comprising:
a first joint part having both penetrated ends, which is detachably coupled to a screw head fixed to a vertebra so as to be exposed to a predetermined height through an opening formed in a skin of a subject; and
a second joint part having both penetrated ends, which is coupled to an upper end of the first joint part and which is linearly movable in a direction that is away from the upper end of the first joint part and rotates to be bent with respect to the upper end of the first joint part after the second joint part is inserted through the opening in a state of being disposed in a straight line together with the first joint part,
wherein the bending of the second joint part at an angle of about 90 degrees or less in both left and right directions with respect to the upper end of the first joint part is restricted after moving in the direction that is away from the upper end of the first joint part in the state of being disposed in the straight line together with the first joint part, and
the second joint part further comprises:
a rotation-allowable cutoff surface cut in a flat shape from the edge of the upper end of the first joint part and formed on each of both outer surfaces of the upper end of the first joint part; and
an excessive rotation restriction protrusion protruding to be stepped on each of both sides of an edge of a lower end of the rotation-allowable cutoff surface, wherein, when the second joint part rotates with respect to the upper end of the first joint part, an edge of one side of each of the rotation pieces extending from both sides of the edge of the lower end of the second joint part contacts the excessive rotation restriction protrusion so that the second joint part is perpendicular to the first joint part.

3. A spinal screw holder for a minimal invasive surgery, comprising:
a first joint part having both penetrated ends, which is detachably coupled to a screw head fixed to a vertebra so as to be exposed to a predetermined height through an opening formed in a skin of a subject;
a second joint part having both penetrated ends, which is coupled to an upper end of the first joint part and which is linearly movable in a direction that is away from the upper end of the first joint part and rotates to be bent with respect to the upper end of the first joint part after the second joint part is inserted through the opening in a state of being disposed in a straight line together with the first joint part;
a rotation-allowable guide groove vertically recessed by a predetermined length in each of both sides of an upper portion of an outer surface of the first joint part;
rotation pieces respectively extending from both sides of an edge of a lower end of the second joint part to face each other;
a rotation pin inserted and fixed from a through-hole passing through each of the rotation pieces and accommodated in the rotation-allowable guide groove; and
a linear movement guide protrusion disposed on each of both sides of the rotation piece in a longitudinal direction of a main body of the first joint part so that the rotation piece linearly moves without rotating when the second joint part moves downward to be coupled.

4. The spinal screw holder of claim 3, wherein an upper end groove having a predetermined width and depth is formed in an upper circumference of the upper end of the first joint part, and
a groove coupling part fitted into the upper end groove to fix the first joint part to the second joint part when being coupled to the upper end of the first joint part is provided in an inner circumference of a lower end of the second joint part.

5. The spinal screw holder of claim 4, wherein a lower end groove having a predetermined width and depth is formed in the inner circumference of the lower end of the second joint part,
the groove coupling part is fixed and installed to the lower end groove in a ring shape, and
when the second joint part is coupled to the upper end of the first joint part, the ring is fitted into the upper end groove to fix the first joint part to the second joint part.
